(19) United States
(12) Patent Application Publication
(10) Pub. No.: US 2020/0319186 A1
(43) Pub. Date: Oct. 8, 2020

(54) MARKERS FOR DIAGNOSIS AND PROGNOSTIC PREDICTION OF NPC AND APPLICATION THEREOF

(71) Applicant: SUN YAT-SEN UNIVERSITY CANCER CENTER (SYSUCC), Guangzhou (CN)

(72) Inventors: Musheng ZENG, Guangzhou (CN); Shibing LI, Guangzhou (CN); Yanyan LIU, Guangzhou (CN); Jianhua SUI, Guangzhou (CN); Huiyu LI, Guangzhou (CN)

(73) Assignee: SUN YAT-SEN UNIVERSITY CANCER CENTER (SYSUCC), Guangzhou (CN)

(21) Appl. No.: 16/766,981

(22) PCT Filed: Apr. 13, 2018
(86) PCT No.: PCT/CN2018/083007

§ 371 Date: May 26, 2020

(30) Foreign Application Priority Data
Feb. 13, 2018 (CN) 201810148513.5

(51) Int. Cl. G01N 33/574; G01N 1/38

(52) U.S. Cl. CPC G01N 33/57407; G01N 2333/62; G01N 1/38

(57) ABSTRACT

Disclosed are markers for nasopharyngeal carcinoma (NPC) diagnosis and prognostic prediction, and use thereof. The INSL5 level in a plasma sample can be determined by means of ELISA. It was found that the INSL5 plasma level is significantly different between NPC patients and healthy population. The analysis results of a ROC curve show that an area under curve of INSL5 is 0.941, the critical value of the INSL5 level is 2.45 ng/ml, and the sensitivity and specificity thereof are 93.2% and 81.5% respectively. In the healthy cohort, the INSL5 level in EBV-positive plasma is significantly higher than that in EBV-negative plasma. The EBV-negative subjects comprise 34 healthy individuals and 72 NPC patients, and the INSL5 levels are observed to be significantly different between these two cohorts. The results of the ROC curve show that the area under curve of INSL5 is 0.988, the critical value of the INSL5 level is 2.25 ng/ml, and the sensitivity and specificity thereof are 97.2% and 91.2% respectively. Thus, the present disclosure can efficiently distinguish EBV-negative normal people from NPC patients.

### CROSS-REFERENCE TO RELATED APPLICATIONS

This application is the United States National Stage entry under 35 U.S.C. 371 of International Application PCT/CN2018/083007 filed on Apr. 13, 2018, which claims the benefit of Chinese Patent Application No. 201810148513.5, filed on Feb. 13, 2018, the entirety of which is incorporated by reference herein.

### TECHNICAL FIELD

The present disclosure is related to the field of medical diagnosis, particularly to the markers for Nasopharyngeal carcinoma (NPC) diagnosis and prognostic prediction, and the use thereof.

### BACKGROUND

Nasopharyngeal carcinoma (NPC) is one of the most common malignant tumors in China, particularly in Southern China, such as Guangdong, Guangxi, Hunan, Fujian and Jiangxi provinces. In addition, NPC is 2-3 times more prevalent in males than females. The reported cases in China mostly fall in people aged 3 to 90, with a high incidence for people aged 30 to 50.

NPC patients often have high antibody titers against Epstein-Barr virus (EBV) in vivo, and have antibody levels which tend to elevate with the progression of the disease. Thus, antibody based stable serum markers are gaining importance for screening and diagnosing NPC positive patients. In the early days, immunofluorescence assay (IFA) was developed and applied as a golden standard technique for screening NPC, but later it was found to be not very effective and consistent in the detection of an EBV capsid antigen (VCA) and an Early Antigen (EA) in serum. In addition, traditional IFA is subjective, time-consuming, and requires high proficiency for technicians, and have varied results with different technicians. Thus, it is difficult to perform quality control. Meanwhile, such assay is less sensitive, and is inefficient to detect patients with EBV-negative nasopharyngeal carcinoma. These limitations were successfully overcome by an enzyme-linked immunosorbent assay (ELISA) which was developed subsequently as a second-generation serological examination technique for nasopharyngeal carcinoma. ELISA is sensitive and convenient, and can perform rapid diagnosis by means of kits, thereby being more suitable for large-scale population screening.

EB viral capsid antigen (VCA) is used as a target for screening and early diagnosis of NPC individuals. Both VCA-IgA IFA and VCA-IgA ELISA are still considered as a first-line assistant diagnostic index for NPC, but the high specificity and sensitivity made ELISA as exceptionally unique for detection of VCA.

Early detection, diagnosis, and treatment of NPC are the key to improving survival rates. VCA-IgA was found positive in 70-95% and 10% of the NPC and normal population, respectively. About 5-30% of the NPC patients negative to VCA-IgA remain overlooked blindly from early diagnosis, and 10% of the VCA-IgA positive normal population undergoing the process of medical diagnosis unnecessarily. Therefore, it is necessary to develop new diagnostic markers for NPC, which would have especially important clinical value for the diagnosis of VCA-IgA-negative patients and could easily distinguish between VCA-IgA-negative NPC patients and VCA-IgA positive normal individuals.

In addition, the five-year survival rate has reached 90% and more for NPC patients, due to the improvement of the modern radio- and chemotherapy. But, unfortunately, we are still inefficient in early diagnosis of NPC. Thus, the development of novel markers for predicting survival prognosis of NPC patients would contribute to optimizing therapeutic regimen for such patients and further improving the survival rate.

Insulin-like factor 5 (INSL5) is a member of insulin (INS) superfamily. It was first discovered in 1999, by Conklin et al. who were investigating cysteine sequences of B-chain of INS superfamily members using expressed sequence tags (EST) database. The structure of INSL5 is similar to those of the members of the insulin superfamily, and includes one signal peptide (A-chain and B-chain), and C-peptide for linking. The structure of INSL5 is stabilized by two inter disulfide between A- and B-chains and one intra disulfide with in A-chain. INSL5 could convert to a hormone having biological activity, after the cleavage of C-peptide for linking. Active INSL5 having A- and B-chains can bind and activate the receptor through leucine 31 and arginine 35 of the B-chain. However, the function of the B-chain binding to and activating the receptor depends on the spatial structure formed by the bound A- and B-chains. Currently, studies on INSL5 suggest that decreased INSL5 level may lead to the impairment of sperm motility for male mice, estrous cycle dysfunction for female mice, a decreased number of islet B cells, the impairment of glucose tolerance, and the like. Meanwhile, some studies suggest that INSL5 may be used as a marker for neuroendocrine tumor. However, there is no report on the use of INSL5 in NPC.

### SUMMARY

One of the objectives of the present disclosure is to provide a suitable and stable blood marker for NPC diagnosis and prognostic prediction, and the use thereof.

The inventors detected the INSL5 level in plasma samples by means of ELISA, and found it has statistically significant difference between NPC patients and healthy individuals.

The analysis results of Receiver operating characteristic (ROC) curve shows that an area under curve (AUC) for INSL5 is 0.941, the critical value for the INSL5 level is 2.45 ng/ml, and the sensitivity and specificity thereof were 93.2% and 81.5% respectively. In the healthy cohort, the INSL5 levels in EBV-positive plasma are significantly higher than those in EBV-negative plasma samples. In 106 EBV-negative individuals, we found 34 cases of healthy people, and 72 cases of NPC patients with statistically significant increase in the INSL5 level. The analysis results of ROC curve showed that the area under curve for INSL5 was 0.988, with the critical value for the INSL5 level was 2.25 ng/ml, and the sensitivity and specificity thereof were 97.2% and 91.2% respectively. Thus, INSL5 may efficiently distinguish EBV-negative normal people from NPC patients.

Regarding prognostic prediction of NPC, Kaplan-Meier survival analysis shows significant differences in both overall survival (OS) and Disease-free survival (DFS) between patients having high- and low-concentrations of INSL5. The concentration critical value of INSL5 was 3.73 ng/ml. If the concentration of INSL5 was higher than 3.73 ng/ml, the five-year survival rate of the patients would be 81.3%, and the incidence rate of distant metastasis would be 11.8%. If the concentration of INSL5 was lower than 3.73 ng/ml, the five-year survival rate of the patients would be 92.2%, and the incidence rate of distant metastasis would be 2.7%. Furthermore, when combining the concentration of INSL5 with the EBV DNA copy number in plasma, it was found that the overall survival and Disease-free survival of NPC patients could be better predicted when the concentration of INSL5 was higher than 3.73 ng/ml and the EBV DNA copy number was higher than 4000 copies/ml.

Therefore, the present disclosure provides a novel detection marker with high sensitivity and specificity for NPC diagnosis and prognostic prediction. Especially, the marker of the present disclosure has good diagnostic value for EBV-negative patients. Therefore, the present disclosure provides a new approach for the screening and diagnosis, as well as the determination and prognostic prediction for NPC.

The technical solutions adopted by the present disclosure are as the following.

Provided is use of INSL5 as a marker for NPC diagnosis and prognostic prediction.

Provided is use of a reagent for quantifying plasma concentration of INSL5 in the preparation of an agent for NPC diagnosis and prognostic prediction.

As an improvement for the above use, the reagent for quantifying the plasma concentration of INSL5 may be selected from INSL5-specific ELISA detection reagents.

As a further improvement for the above use, the criterion for high risk of NPC can be determined by plotting a ROC curve, and the criterion for poor NPC prognosis can be determined by a Kaplan-Meier survival analysis.

As a further improvement for the above use, the criterion for poor prognosis of nasopharyngeal carcinoma can be determined by the Kaplan-Meier survival analysis with combining INSL5 with the plasma EBV copy number.

Provided a method for diagnosis and prognostic prediction of nasopharyngeal carcinoma, which may comprise the following steps:

1) detecting an INSL5 concentration in a plasma sample to be tested;

2) categorizing the sample for predisposition to NPC based on the detected concentration of INSL5, to determine whether the sample to be tested is a high-risk sample of NPC and/or predicting prognosis thereof.

As an improvement for the above method, the criterion for high risk of nasopharyngeal carcinoma can be determined by plotting a ROC curve, and the criterion for poor prognosis of nasopharyngeal carcinoma can be determined by a Kaplan-Meier survival analysis, preferably by the Kaplan-Meier survival analysis with combining INSL5 with the plasma EBV DNA copy number.

As a further improvement for the above method, the Yuden index of the ROC curve may be a high risk criterion for NPC.

As a further improvement for the above method, the concentration of INSL5 higher than 2.45 ng/ml may be a high risk criterion for NPC.

The concentration of INSL5 higher than 3.73 ng/ml, or the concentration of INSL5 higher than 3.73 ng/ml in combination with the EBV DNA copy number higher than 4000 copies/ml, may be a criterion for poor prognosis of NPC.

As a further improvement for the above method, the step for determining the concentration of INSL5 may comprise:

1) diluting a plasma sample in a ratio of 1:10 with a sample diluent, adding the sample in a microplate 100 μl/well, and incubating for 2 hours at 37° C.;

2) washing the plate with 200 μl PBST for three times, then removing PBST by flicking the plate over a sink and patting the plate on a paper towel and adding a diluted biotin-labeled detection antibody 23G9 100 μl/well, and incubating for 2 hours at 37° C.;

3) washing the plate with 200 μl PBST for three times, then removing PBST by flicking the plate over a sink and patting the plate on a paper towel and adding diluted streptavidin conjugated with HRP 100 μl/well, and incubating for 20 minutes in dark at room temperature;

4) washing the plate with 200 μl PBST for five times, then removing PBST by flicking the plate over a sink and patting the plate on a paper towel and adding a chromogenic substrate TMB 100 μl/well, and developing for no more than 20 minutes at 37° C.;

5) terminating the reaction by adding 50 μl of 2M H2SO4 and standing for 10 min;

6) reading Optical density (OD) values at wavelength of 450 nm on a microplate reader, and calculating the concentration of INSL5 in the sample to be tested according to a standard curve.

The present disclosure has the following advantages.

The detection kit provided by the present disclosure has advantages such as easy to use, high reproducibility, sensitivity and specificity. Experimental data shows that the concentration of INSL5, taking a critical value of 2.45 ng/ml, can be used to effectively distinguish normal individuals from NPC patients, and the sensitivity and specificity are 93.2% and 81.5% respectively. In addition, the concentration of INSL5, taking a critical value of 2.25 ng/ml, can be used to effectively distinguish EBV-negative normal individuals from EBV-negative NPC patients, and the sensitivity and specificity are 97.2% and 91.2%, respectively.

In addition, INSL5 may be used as a prognostic prediction marker for NPC, with a critical value of 3.73 ng/ml. If the concentration of INSL5 is higher than 3.73 ng/ml, it would indicate poor prognosis, and the five-year overall survival rate and Disease-free survival rate are 81.3% and 88.2% respectively, which are much lower than the patients with lower INSL5 concentration who have five-year survival rate of 92.2% and the five-year Disease-free survival rate of 97.3%. Meanwhile, the prognosis of NPC patients can be better predicted by combining the concentration of INSL5 with the EBV DNA copy number (with a critical value of 4000 copies/ml).

In view of the above, the present disclosure provides a new highly sensitive and specific detection marker for the diagnosis and prognostic prediction of NPC, and thus can be beneficial to the diagnosis and therapeutic monitoring of NPC.

### BRIEF DESCRIPTION OF THE DRAWINGS

FIG. 1 shows diagrams confirming that INSL5 antibodies 46B8 and 23G9 can bind INSL5 and have different epitopes, and thus can be used for sandwich ELISA to detect INSL5.

FIG. 2 is a scatter plot showing the concentrations of INSL5 in NPC patients, healthy control cohort with EBV-positive (Normal EBV(+)) and healthy control cohort with EBV-negative (Normal EBV(−)).

FIG. 3 shows the results of ROC curve analysis of the concentrations of INSL5 in NPC patients and healthy control cohort (normal).

FIG. 4 is a ROC curve which shows the diagnostic efficacy of INSL5 for EBV-negative NPC patients.

FIG. 5 is a curve which shows the results of survival prognosis analysis by means of INSL5 alone;

FIG. 6 is a curve which shows the results of survival prognosis analysis by means of the combination of INSL5 and the EBV DNA copy number.

### DETAILED DESCRIPTION

Preparation of INSL5 Antibodies.

BALB/c female mice of 6-8 weeks old were routinely immunized by intraperitoneal injection of a recombinantly expressed mature INSL5 fusion protein having a human Fc tag, in 50 μg/mouse/time, following by strengthening immunization every two weeks. After three times of immunization, the antibody titers in the mouse serum was determined by ELISA. Then, the mice having higher antibody titers were sacrificed after strengthening immunization once. The spleens were taken out for making cell suspension, which then fused with SP2/0 mouse myeloma cells. The antigen, which was used for the detection of antibody titers of mouse serum by ELISA, was a biotinylated mature INSL5-His fusion protein containing a 6×His tag. After cell fusion, a lot of ELISA screening and subcloning were performed, and finally obtained two clones, 46B8 and 23G9, which showed stronger specific binding activity to the mature INSL5-His fusion protein. In addition, the two clones, 46B8 and 23G9, could bind to Pre-INSL5, in which the linking peptide is not completely removed. It shows that the binding epitopes of the two clones locate on A or B chain of INSL5 protein.

Buffers and Reagents for the INSL5 ELISA Test Kit.

ELISA plate: a microplate pre-coated with the antibody 46B8 for capturing INSL5. The preparation method thereof was as follows:

1) diluting INSL5 antibody 46B8 with a coating buffer to 2 μg/ml, and adding 100 μl of the diluted 46B8 antibody into each well of the microplate, and incubating at room temperature overnight;

2) spin-drying the coating buffer, washing 3 times with PBST, then removing PBST and adding a blocking solution containing 3% BSA, 300 μl/well, and blocking at room temperature for 2 hours;

3) washing the plate with PBST for 3 times, patting and removing PBST, and storing at 4° C. until use.

Coating buffer: PBS buffer, pH 7.3;

Blocking solution: 3% BSA, 1×PBS;

Diluent: 1×PBS, pH 7.2-7.4;

Washing solution: 1×PBS, 0.05% Tween-20, pH 7.2-7.4;

Standard samples: INSL5 powder was dissolved and diluted to obtain a gradient concentration of solutions, as the standard samples, which can be purchased from Phoenix Biotech;

Detection antibody: 23G9 which was labeled with biotin purchased from Thermofisher;

Detection reagent: HRP-conjugated streptavidin, purchased from R&D

Corporation;

Chromogenic substrate: TMB, purchased from Sigma;

Elimination solution: 2M H2SO4.

Patients with Nasopharyngeal Carcinoma.

339 cases of inpatients and outpatient with NPC were collected from Sun Yat-sen University Cancer Center, from January 2009 to December 2012. Inclusion criteria was that the patients had been confirmed to have type II/III nasopharyngeal carcinoma by pathological diagnosis (based on World Health Organization Classification System 1978) and had precise clinical stages (based on USA AJCC cancer staging manual, 7th edition); all the samples were derived from the patients from the areas having high incidence of nasopharyngeal carcinoma in southern China (e.g., Guangdong, Guangxi, Jiangxi, Hunan, Fujian, and Sichuan provinces); the plasma samples were taken before treatment; 284 of the 339 cases had complete demographic data and medical records, including the results of serum VCA-IgA titer and quantitative detection of plasma EBV DNA copies. Among the patients, there were 75 EBV-negative cases and 209 EBV-positive cases.

Healthy Population.

65 plasma samples were randomly selected from the physical examination of staffs of Sun Yat-Sen University. The data, including the results of VCA-IgA titer, of all the healthy population were kept, with the exclusion of other diseases. Among them, there were 34 EBV-negative cases and 31 EBV-positive cases.

Detection Process.

The detection process comprised:

1) diluting the plasma samples in the ratio of 1:10 with a sample diluent, adding the samples 100 μl/well, and incubating for 2 hours at 37° C.;

2) washing the plate with PBST for three times, then removing PBST by flicking the plate over a sink and patting the plate on a paper towel and adding the diluted biotin-labeled detection antibody 23G9 100 μl/well, and incubating for 2 hours at 37° C.;

3) washing the plate with PBST for three times, then removing PBST by flicking the plate over a sink and patting the plate on a paper towel and adding the diluted streptavidin conjugated with HRP 100 μl/well, and incubating for 20 minutes in dark at room temperature;

4) washing the plate with PBST for five times, then removing PBST by flicking the plate over a sink and patting the plate on a paper towel and adding the chromogenic substrate TMB100 μl/well, and developing at 37° C. for no more than 20 minutes;

5) terminating the reaction by adding 50 μl of 2M H2SO4 and standing for 10 min;

6) reading OD values at wavelength of 450 nm on the microplate reader, and calculating the concentrations of INSL5 in the samples to be tested according to the standard curve.

Detection Results and Analysis.

1. Antibody Verification

The antibodies 46B8 and 23G9 against INSL5 were demonstrated to have different epitopes by competitive ELISA, thereby being capable of assembling an ELISA Kit for the detection of INSL5.

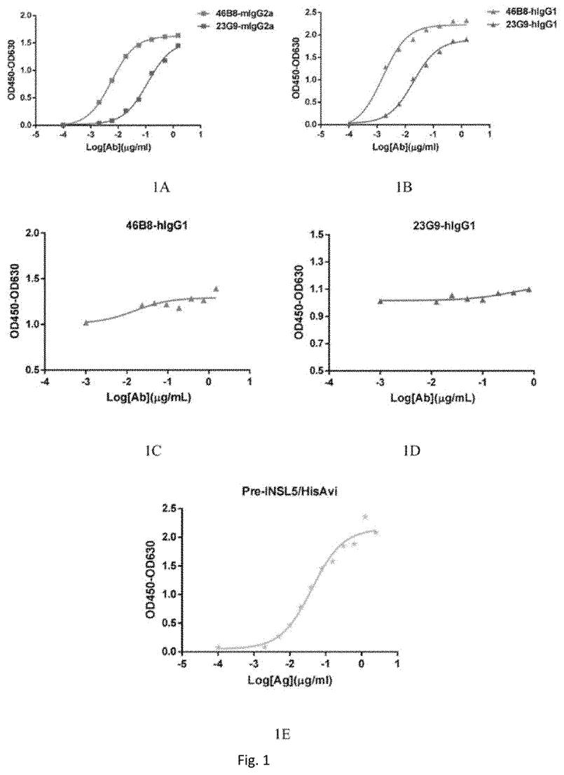

Mouse antibodies 46B8-mlgG2a and 23G9-mlgG2a, as well as human-mouse chimeric antibodies 46B8-hIgG1 and 23G9-hIgG1 were recombinantly expressed by the inventors. As shown in FIGS. 1A and 1B, the four recombinantly expressed antibodies had good binding ability to the antigen Pre-INSL5 protein. Then, it was found that 46B8-hIgG1 could not compete for the binding of 23G9-mlgG2a to Pre-INSL5 antigen (FIG. 1C), and 23G9-hIgG1 also could not compete for the binding of 46B8-mlgG2a to Pre-INSL5 antigen (FIG. 1D). Further, it could detect different INSL5 of different concentrations by using 46B8 as a capture antibody and 23G9 as a detection antibody (FIG. 1E).

2. Detection of Plasma Samples.

339 cases of NPC patients and 65 cases of healthy controls were selected for the detection of plasma samples which were derived from Sun Yat-sen University Cancer Center. The concentrations of INSL5 were calculated according to the concentrations of the standard samples, and analyzed by SPSS (20.0) statistical software. Statistical analysis showed that the data was non-normal distribution, and thus was analyzed by the Wilcoxon rank sum test.

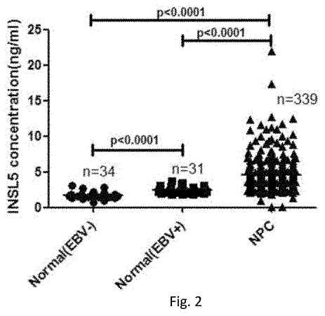

The results of the Wilcoxon rank sum test showed that there was a statistically significant difference in the plasma concentrations of INSL5 between the two cohorts of population. The INSL5 plasma concentrations of NPC patients were significantly higher than those of the normal cohort (p value is less than 0.0001). Meanwhile, the INSL5 concentrations of the EBV-positive cohort were significantly higher than those of the EBV-negative cohort in the normal cohort, and the scatter diagram thereof was shown in FIG. 2.

TABLE 1
Comparison of INSL5 levels between
NPC patients and normal cohorts

| | Median concentration | Interquartile | P |
|---|---|---|---|
| Cohorts | (ng/ml) | range | value |
| | | | <0.001 |
| Nasopharyngeal | 4.1464 | 1.886 | |
| carcinoma | | | |
| Normal EBV (+) cohort | 2.2854 | 0.7343 | |
| Normal EBV (−) cohort | 1.6524 | 0.3292 | |

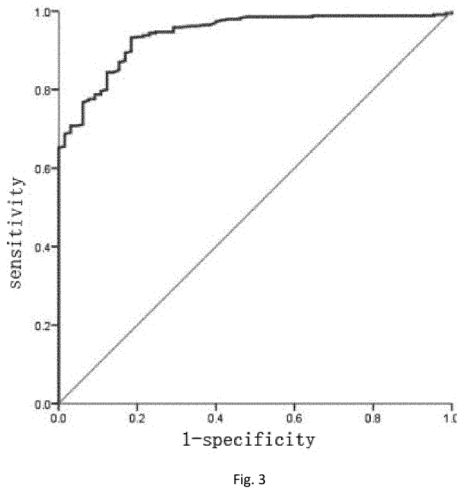

A ROC curve was plotted based on the INSL5 concentrations of the NPC patients and the normal cohort (FIG. 3).

The Cut-off value was set based on the concentration of INSL5 when Yoden index reached the maximum value. In the way, the concentration of INSL5 of 2.45 ng/ml was selected as the Cut-off value based on the analytical curve. The detection sensitivity was 93.2%, the detection specificity was 81.5%, and the area under curve was 0.941, when the concentration of INSL5 was used for the NPC diagnosis.

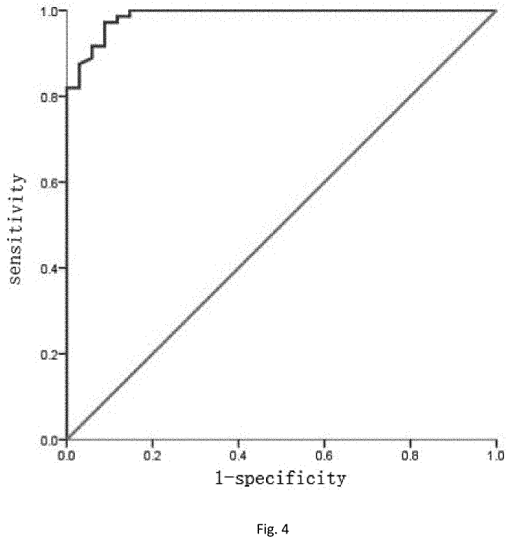

In addition, INSL5 can clearly distinguish the NPC patients from normal people, in the EBV-negative population. A ROC curve was plotted based on the INSL5 concentrations in the EBV-negative cohort (FIG. 4). The cut-off value was set be the concentration of INSL5 when the Yoden index reached the maximum value. In this way, the concentration of INSL5 of 2.25 ng/ml was selected as the Cut-off value based on the analytical curve. The detection sensitivity was 97.2%, the detection specificity was 91.2%, and the area under curve was 0.988, when the INSL5 concentration was used for the NPC diagnosis.

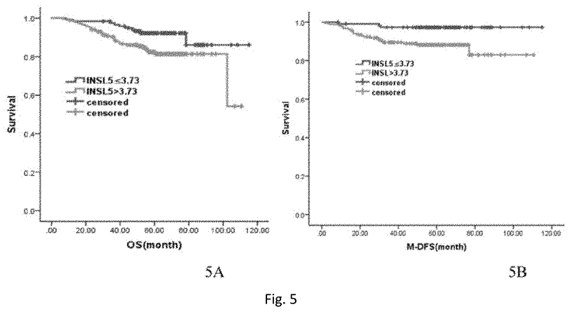

With the analysis of 304 cases having prognostic information, it was found that INSL5 was an independent prognostic factor, with a critical value of 3.73 ng/ml. A higher concentration of INSL5 can predict poor prognosis. The five-year survival rate and the Disease-free survival rate were 81.3% and 88.2% respectively, which were much lower than those in the cohort with lower INSL5 concentration which had the five-year survival rate of 92.2% and Disease-free survival rate of 97.3% (FIG. 5).

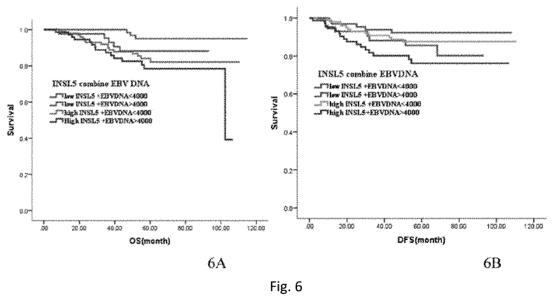

In addition, the prognosis of NPC patients could be better predicted by the combination of the concentration of INSL5, and the EBV DNA copy number which has a critical value of 4000 copies/ml (FIG. 6).

In conclusion, the experimental data shows that quantitative analysis of the INSL5 plasma concentration can provide a new detection marker which has high sensitivity and specificity for the NPC diagnosis, especially can be valuable for the diagnosis of EBV-negative patients and reduce the possibility of missed diagnosis. Meanwhile, INSL5 can be used as a good independent marker for prognostic prediction, and INSL5 used in combination with the EBV DNA copy number can improve the ability of prognostic prediction. Thus, the present disclosure provides a new approach for the NPC diagnosis and prognostic prediction.

What is claimed is:

1. A method for nasopharyngeal carcinoma (NPC) diagnosis and prognostic prediction, said method comprising: using Insulin-like factor 5 (INSL5) as a marker.

2. A method for nasopharyngeal carcinoma (NPC) diagnosis and prognostic prediction, said method comprising: using a reagent for quantifying a plasma concentration of Insulin-like factor 5 (INSL5) in the preparation of an agent.

3. The method of claim 2, wherein the reagent for quantifying the plasma concentration of Insulin-like factor 5 (INSL5) is selected from INSL5-specific ELISA detection reagents.

4. The method of claim 2, wherein a criterion for high risk of nasopharyngeal carcinoma (NPC) is determined by plotting a ROC curve, and a criterion for poor prognosis of nasopharyngeal carcinoma is determined by a Kaplan-Meier survival analysis.

5. The method of claim 4, wherein the criterion for poor prognosis of NPC is determined by the Kaplan-Meier survival analysis with combining INSL5 with an EBV DNA copy number in plasma.

6. A method for nasopharyngeal carcinoma (NPC) diagnosis and prognostic prediction, said method comprising:
determining an INSL5 concentration in a plasma sample to be tested;
determining whether the plasma sample to be tested is a high-risk sample of NPC; and
predicting prognosis thereof based on the determined INSL5 concentration.

7. The method of claim 6, further comprising:
determining a criterion for high risk of NPC by plotting a ROC curve, and
determining a criterion for poor prognosis of NPC through either a Kaplan-Meier survival analysis, or through the Kaplan-Meier survival analysis with combining INSL5 with a EBV DNA copy number in plasma.

8. The method of claim 7, wherein the criterion for high risk of NPC is a Yuden index of the ROC curve.

9. The method of claim 7, wherein,
determining to have high risk of NPC when the INSL5 concentration is higher than 2.45 ng/ml;
determining to have poor prognosis of NPC when the INSL5 concentration is higher than 3.73 ng/ml, or when the INSL5 concentration is higher than 3.73 ng/ml and a EBV DNA copy number is higher than 4000 copies/ml.

10. The method of claim 6, wherein the step of determining the INSL5 concentration comprises:
diluting the plasma sample in a ratio of 1:10 with a sample diluent, adding the plasma sample in a microplate 100 μl/well, and incubating for 2 hours at 37° C.;
washing the plate with PBST for three times, then removing PBST and adding a diluted biotin-labeled detection antibody 23G9 100 μl/well, and incubating for 2 hours at 37° C.;
washing the plate with PBST for three times, then removing PBST and adding a diluted streptavidin conjugated with HRP 100 μl/well, and incubating for 20 minutes in dark at room temperature;
washing the plate with PBST for five times, then removing PBST and adding a chromogenic substrate TMB 100 μl/well, and developing for no more than 20 minutes at 37° C.;
terminating the reaction by adding 50 μl of 2M H₂SO₄ and standing for 10 min; and
reading an Optical density (OD) value at wavelength of 450 nm on a microplate reader, and calculating the INSL5 concentration in the sample to be tested according to a standard curve.

11. A method for nasopharyngeal carcinoma (NPC) diagnosis and prognostic prediction, said method comprising:
determining an INSL5 concentration in a plasma sample to be tested; and
determining whether the plasma sample to be tested is a high-risk sample of NPC or predicting prognosis thereof based on the determined INSL5 concentration.
